(19) United States
(12) Patent Application Publication
(10) Pub. No.: US 2014/0148826 A1
(43) Pub. Date: May 29, 2014

(54) MINIMALLY INVASIVE SURGICAL KIT

(71) Applicant: PERGOLAS, LLC, Cupertino, CA (US)

(72) Inventors: Gary Chorng-Jyh Wang, Cupertino, CA (US); Wilson Wen-Fu Wang, San Jose, CA (US); Chen-Chie Wang, New Taipei City (TW); Jenyang Yu, Cupertino, CA (US)

(73) Assignee: PERGOLAS, LLC, Cupertino, CA (US)

(21) Appl. No.: 13/687,886

(22) Filed: Nov. 28, 2012

(51) Int. Cl. A61B 17/04

(52) U.S. Cl. CPC A61B 17/0469
USPC 606/148

(57) ABSTRACT

A surgical kit comprising a suture retriever comprising a handle element coupling with an extending element, wherein the extending element is coupled with a hook portion; wherein the hook portion comprises a spiral structure and a suture collector comprising suture collecting elements forming one or more slits configured to receive a suture from the suture retriever and a base coupling both sides of the suture collecting elements.

### CROSS-REFERENCE TO RELATED APPLICATION(S)

This application claims priority to the Taiwanese Patent Application Ser. No. 10046447, filed Dec. 15, 2012 and titled, “Surgical Kit,” which is also hereby incorporated by reference in its entirety for all purposes.

### FIELD OF THE INVENTION

The present invention relates to surgical devices. More specifically, the present invention relates to a minimally invasive surgical kit.

### BACKGROUND OF THE INVENTION

The traditional surgery approach performs destruction first and then rebuild. The wound produced by this approach is generally bigger and the patient experience greater pain and longer recovery period. In recent years, with the advancement of technology, minimally invasive surgery, such as endoscopic surgery and laser surgery, has been widely used, thus greatly reduce the pain from the surgery.

A goal of the minimally invasive surgery is to minimize the injury and surgical wound to help a patient to recover quickly. Using a spine surgery as an example, a spine surgery should avoid damage to the bones, ligaments, fascia, muscle and blood vessels in order to maintain the normal functions (stabilize the normal body movements) of the spine. If a spine or the neighboring tissue is damaged during the operation, it has greater opportunity to affect the function of the surrounding tissues and furthermore to cause other issues or symptoms. Accordingly, a minimally invasive surgery is ideal to minimize the surgical wound and keep the tissues and muscles untouched.

Endoscopy surgery is a very important part of the minimally invasive surgery. With the advances of the technology, endoscopy surgery is used to treat issues like bone spurs, yellow ligament hypertrophy, facet joint hypertrophy, causing nerve root compression, neural foramen stenosis spinal stenosis cause numbness of the arm, shoulder numbness, numbness of the neck, lower back pain, hip pain, leg tingling pain, sciatica, leg and foot weakness or flexible. Such surgery is able to be performed with a local anesthesia, need only a short period of operation time, and result in a small surgical wound, which result in a shortened hospital stay.

Some elderly patients with chronic conditions, such as hypertension, diabetes, cardiac arrhythmia, high blood cholesterol, or stroke, require a general anesthesia during a traditional or microscopic surgery, which has a higher risk when it is compared to a local anesthesia in a minimally invasive surgery.

In the minimally invasive surgery with local anesthesia, a patient can stay conscious to interact with surgeons and report any discomfort so that surgeons are able to respond immediately.

One of the advantages of using a minimally invasive surgery is that it results in a smaller surgical wound, which cause less damage to the body and thus reduces the time for hospital stay and recovery period. The minimally invasive surgery is able to minimize the complications caused by the surgery and allows patients to return to the normal daily life quickly after the surgery. Although minimally invasive surgery is able to result in a smaller surgical wound, it requires sophisticate tools.

### SUMMARY OF THE INVENTION

The present invention provides a minimally invasive surgical kit, which comprises a suture retriever and a suture collector. The suture retriever comprises a handle element and a hook portion. One end of the handle element extends as an extending element. One end of the hook portion connects with the extending element and another end of the hook portion bends inward as a G-shape structure. An outer side of the hook portion has a bluntness surface. The suture retriever provides many advantages aspects. For example, wounds results from the surgical operation are able to be smaller than one centimeter. The suture collector comprises a substrate formed as a frame structure and a collection body having a plurality of suture clamp portions. Both ends of the collection body are connected/coupled with the substrate. A gap is formed in between each of the clamp portions as suture receivers by connecting at the root of the plurality of clamp portions. After the surgical retriever is taken out from a surgical site, at least one of the sutures is collected in one of the gaps of the suture collector.

In some embodiments, the extending element and the handle element form a 5 to 15 degree angle. In some embodiments, the extending element has a rectangular shape with a thin thickness. In some embodiments, the thickness of the hook portion is similar to the thickness of the extending element, such as 0.5 cm. In other embodiments, the thickness of the hook portion and the thickness of the extending element is 0.3 to 0.7 cm. In some embodiments, the hook portion comprises a straight shank element, a first bending element, a first extending element, a second bending element and a second extending element. In some embodiments, a straight shank element is extended from one side of the extending element. In some embodiments, the first bending element is connected to the straight shank element and bended along the other side of the extending element. In some embodiments, the first extending element is connected to the first bending element and is parallel to the straight shank element. In some embodiments, the second bending element is connected to the first extending element and bended toward the straight shank element. In some embodiments, the second extending element is connected to the second bending element and is parallel to the straight shank element and the first extending element. In some embodiments, the straight shank element, first bending element, first extending element, second bending element and second extending element are tooled/manufactured as one part. In some embodiments, the suture retriever is made by hardened steel, tempered steel, stainless steel, high carbon steel or a composition material. In some embodiments, the clamp portions in the suture collector are form in an inverted-U shape. In some embodiments, a gap formed between each clamp portion is between 0.3 and 0.7 cm. In some embodiments, the suture collector is made by hardened steel, tempered steel, stainless steel, high carbon steel or a composition material.

The surgical kit provides the following advantages: the surgical wound can be less than 1 cm when using the suture retriever; the suture retriever separates the tissues when it is pushed through the body and thus minimizes the damage to the tissues; it shortens the recovery time; the suture collector organizes the sutures used in the surgery. In some embodiments, a single thread suture is used to connect to a needle and each suture has a different color so no two sutures share the same color. Different colors of sutures provide an easy identification system. In other embodiments, a Y-shape cut suture collector is used. The Y-shape cut suture collector is able to be made by a disposable and foldable material such as paper. A suture is able to be clamped between the Y-shape cut. In other embodiments, the suture retriever is made by disposable material for one time use. This is to reduce the cost for sensitization and to reduce the risk of infections caused by the sensitization process.

In a first aspect, a surgical kit comprises a suture retriever comprising a handle element coupling with an extending element, wherein the extending element is coupled with a hook portion; wherein the hook portion comprises a spiral structure and a suture collector comprising suture collecting elements forming one or more slits configured to receive a suture from the suture retriever and a base coupling both sides of the suture collecting elements. In some embodiments, the spiral structure comprises a truncated, a partial, or an abbreviated spiral structure. In other embodiments, the spiral structure comprises a coiled structure. In some other embodiments, the extending element comprises a thin rectangular structure. In some embodiments, the width of the hook portion is the same as the width of the extending element. In some other embodiments, the width is in the range of 0.3 cm to 0.7 cm. In some embodiments, the hook portion comprises a straight shank element extending straight away from the extending element, a first bending element, a first extending element, a second bending element, and a second extending element. In other embodiments, the straight shank element is directly connected to the first bending element bended toward the extending element, wherein the first bending element is directly connected to the first extending element that is extended straight toward the extending element. In some embodiments, the first extending element is parallel to the straight shank element. In some other embodiments, the second extending element is parallel to the straight shank element and the first extending element. In some embodiments, the straight shank element, the first bending element, the first extending element, the second bending element and the second extending element are formed as an integral piece. In some other embodiments, the suture retriever or the suture collector is made by hardened steel, tempered steel, stainless steel, high carbon steel or a composition material. In some embodiments, the extending element has the same thickness as the handle element. In other embodiments, the suture collector is made by a disposable material, a foldable material, or a combination thereof.

In a second aspect, a surgical kit comprises a suture retriever comprising a coiled suture hook on an angled extending arm and a suture collector comprising multiple suture receivers to receive sutures from the suture retriever, wherein the sutures comprise different colors. In some embodiments, the surgical kit further comprises a needle coupling with only one of the sutures.

In a third aspect, a method of using a surgical kit comprises retrieving a suture using a suture retriever, wherein the suture retriever comprises a handle element coupling with an extending element, wherein the extending element is coupled with a hook portion, wherein the hook portion comprises a spiral structure and receiving the suture using a suture collector comprising suture collecting elements comprising one or more slits. In some embodiments, the spiral structure comprises a truncated, a partial, or an abbreviated spiral structure. In other embodiments, the spiral structure comprises a coiled structure. In some other embodiments, the method further comprises placing sutures with different colors on the suture collector.

### BRIEF DESCRIPTION OF THE DRAWINGS

FIG. 1 illustrates a side view of a suture retriever according to some embodiments.

FIG. 2 illustrates a top view of a suture retriever according to some embodiments.

FIG. 3 illustrates a hook portion of the suture retriever according to some embodiments.

FIG. 4 illustrates a suture collector according to some embodiments.

FIG. 5 illustrates a usage of the suture collector according to some embodiments.

FIG. 6 illustrates a needle with a single thread suture according to some embodiments.

FIG. 7 illustrates an Achilles surgery using the surgical kit with color sutures according to some embodiments.

FIG. 8 illustrates a disposable suture collector according to some embodiments.

FIG. 9 illustrates a method of using the surgical kit according to some embodiments.

### DETAILED DESCRIPTION

Embodiments will now be described by way of examples, with reference to the accompanying drawings which are meant to be exemplary and not limiting. For all figures mentioned herein, like numbered elements refer to like elements throughout.

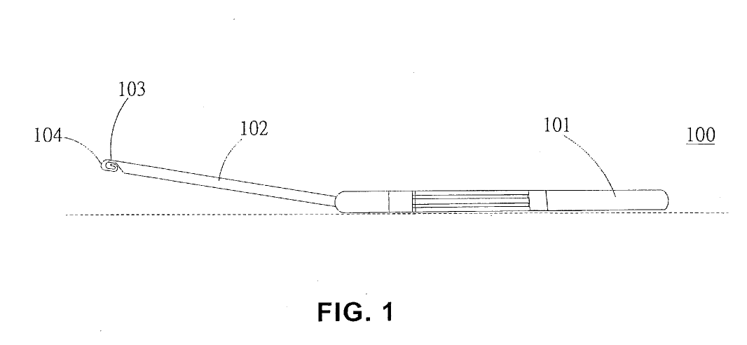

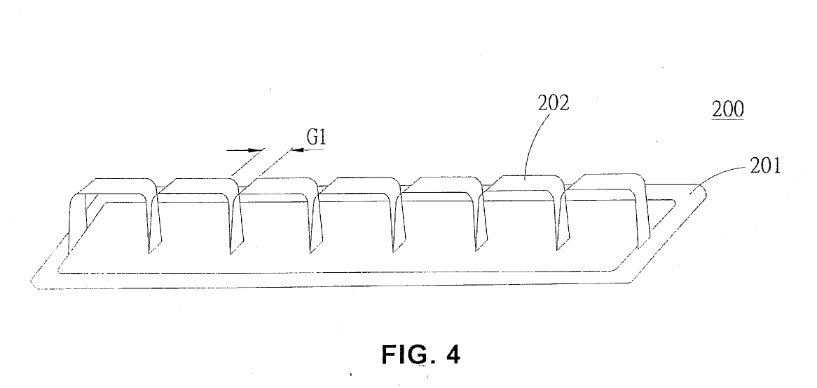

In the following, a surgical kit according to some embodiments is disclosed. In some embodiments, the surgical kit comprises a suture retriever 100 (FIGS. 1-3) and a suture collector 200 (FIG. 4). A side view of the suture retriever 100 is illustrated in FIG. 1. The suture retriever 100 comprises a handle element 101, an extending element 102, and a hook portion 103.

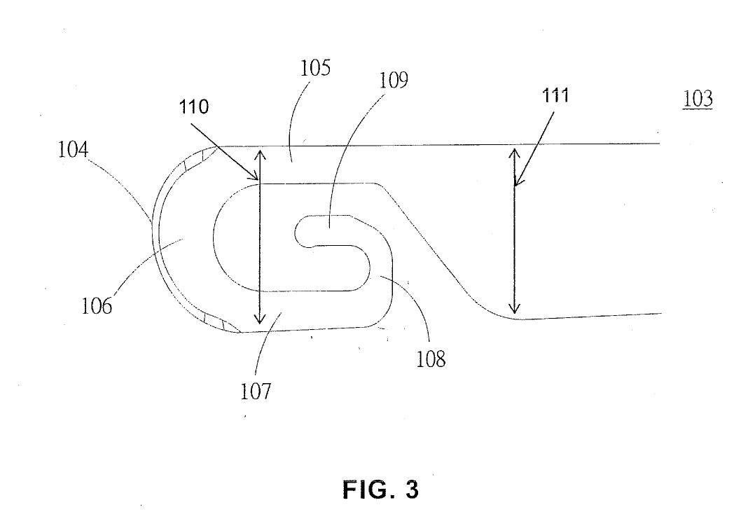

The suture retriever 100 is able to be used to push away the tissues to the side and to retrieve a suture that is inserted in the body. The handle element 101 is able to be connected to or couple with the extending element 102. The extending element 102 is able to be connected to or coupled with the hook portion 103. The hook portion 103 is able to bend inward similar to a G-shape structure and the outside of the hook portion 103 is a bluntness surface 104. In some embodiments, the extending element 102 and the handle element 101 form a 5 to 15 degree angle. The width 110 (FIG. 3) of the hook portion 103 is able to be the same or similar to the width 111 (FIG. 3) of the extending element 102. In some embodiments, the thickness/length/width of the hook portion 103 is able to be between 0.3 cm and 0.7 cm. In some other embodiments, the length/width/thickness of the hook portion 103 is able to be between 0.7 cm and 1.5 cm.

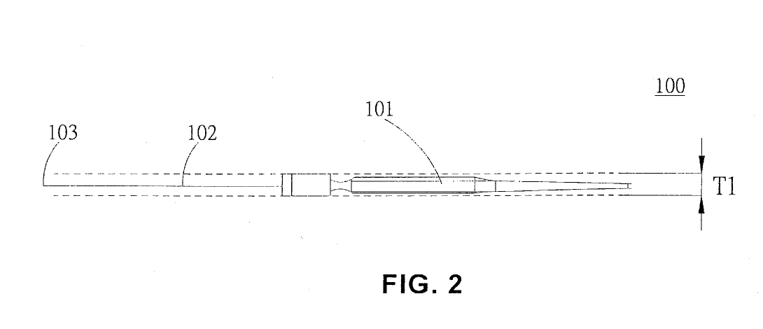

FIG. 2 illustrates a top view of the suture retriever 100 in accordance with some embodiments. In some embodiments, the handle element 101 is designed to be easy gripped and handled by a surgeon. In some embodiments, the extending element 102 is a thin rectangular structure and extends from the handle element 101. In some embodiments, the maximum thickness of T1 is 0.7 cm. In some embodiments, the suture retriever 100 is made by hardened steel, tempered steel, stainless steel, high carbon steel or a composition material. A person of ordinary skill in the art appreciates that any other materials is able to be used to make the suture retriever 100.

FIG. 3 illustrates a close-up view of the hook portion 103 in accordance with some embodiments. A straight shank element 105 is able to be extended straight from one side of the extending element 102 and is connected to a first bending element 106. The first bending element 106 is bended curving toward the extending element 102 and is connected to a first extending element 107 that is extended in a straight line toward the extending element 102. The first extending element 107 is able to be parallel to the straight shank element 105. The other end of the first extending element 107 is connected to/coupled with the second bending element 108. The second bending element 108 is bended curving toward the first bending element 106 and is connected to a second extending element 109. The second extending element 109 is able to be parallel to the straight shank element 105 and the first extending element 107. The straight shank element 105, the first bending element 106, the first extending element 107, the second bending element 108 and the second extending element 109 jointly form a G-shape (partial spiral, spiral, or coiled) structure 103.

In other embodiments, the hook portion 103 (the straight shank element 105, the first bending element 106, the first extending element 107, the second bending element 108, and the second extending element 109) is able to be manufactured, tooled, and produced as one single (inseparable) structure.

FIG. 4 illustrates a suture collector 200 in accordance with some embodiments. In some embodiments, the suture collector 200 comprises the base element 201 and one or more collector elements 202. The suture collector 200 is able to be used to organize the sutures and provide an easy identification for different sutures. The collector elements 202 are connected one after another. The first and the last collector elements are able to be connected to the base element 201. A gap G1 is in between each collector element 202. The gap G1 is able to be in between 0.3 cm and 0.7 cm. In some embodiments, the gap G1 is in the range of 0.45 to 0.55 cm, such that it is able to be used to organize the sutures during a surgery.

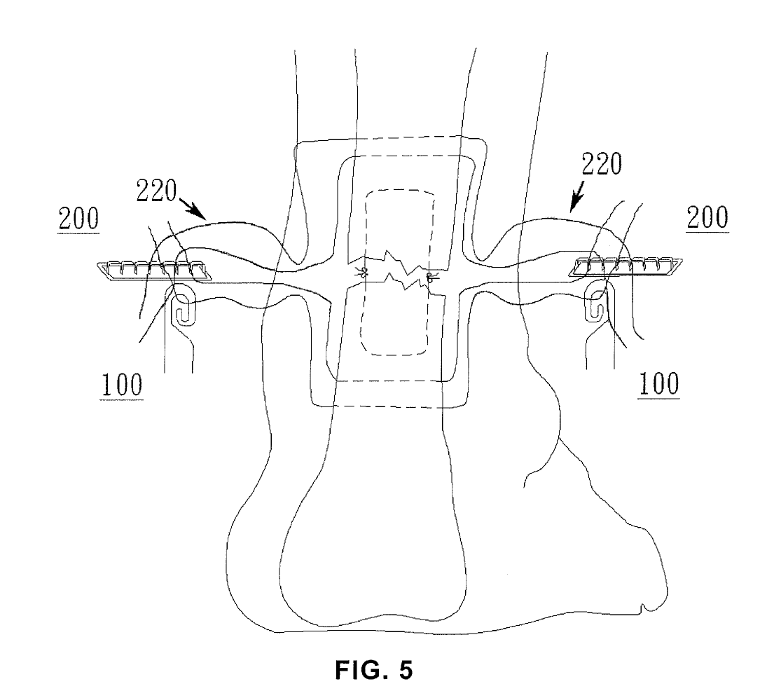

FIG. 5 illustrates a use of the surgical kit in accordance with some embodiments. The suture retriever 100 is able to be pushed through the body without damaging or cutting issues to minimize the wound. After the suture retriever 100 retrieves one or more sutures 220, the sutures 220 are placed in a predetermined/desired order (e.g. by a descending number sequence) in the suture collector 200. This method is able to avoid mixing the sutures 220 and to increase the efficiency of the surgery. In addition, the surgical kit (suture retriever 100 and suture collector 200) allows a single surgeon to perform a surgical procedure alone without the help from an assistant, such that numbers of people needed for the operation is able to be reduced. Space in the surgery room is able to be saved accordingly. The use of the surgical kit also simplifies the surgical procedure when a single surgeon performs the surgery.

The surgical kit has the following one or more advantages: the surgery wound is able to be smaller than 1 cm; the suture retriever is able to minimize the damage to the tissues; the use of the surgical kit is able to shorten the recovery time; the suture collector increases the efficiency of the surgery by organizing the sutures efficiently; single surgeon is able to perform the surgery; and the surgical kit is able to be sensitized using a high temperature and high pressure sterilization process.

In some embodiments, the thickness of the extending element 102, the thickness of the hook portion 103 and the thickness of the handle element 101 are able to be the same (FIG. 1). In some other embodiments, the thickness of the hook portion 103 is different from the thickness of the handle element 101. In some embodiments, the extending element 102 (at the end connecting to the hook portion 103) has the same thickness as the thickness of the hook portion 103. Similarly, the handle element 101 is able to have the same or similar thickness/width as the extending element 102 where the two parts are connected with each other.

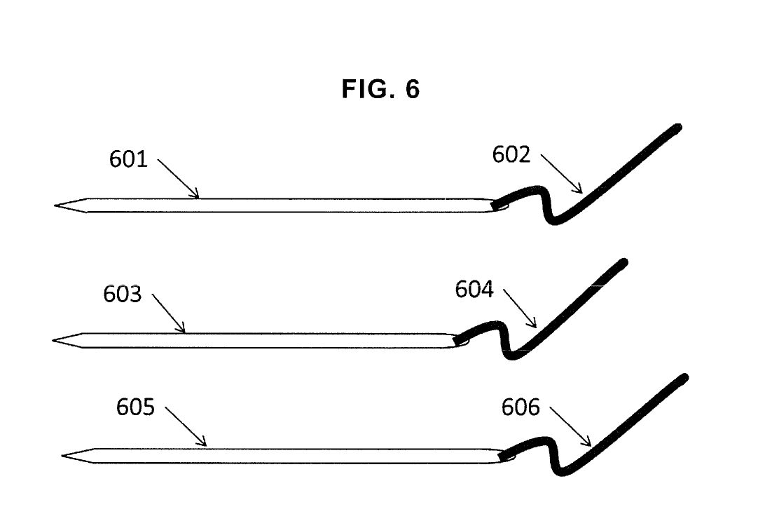

FIG. 6 illustrates the needles and sutures included in the surgical kit in accordance with some embodiments. Needles 601, 603, and 605 and sutures 602, 604, and 606 are able to be constructed/manufactured in one piece. The needles 601, 603 and 605 are able to be used to penetrate through the body during a surgery. Each of the needles 601, 603, and 605 is connected to one single thread of suture. The needle 601 is connected to one single thread of suture 602. The needle 603 is connected to one single thread of suture 604. The needle 605 is connected to one thread of suture 606.

In some embodiments, the length of the needle is able to be 20 cm or longer to be able to penetrate through most of the body parts while some part of the needle remains outside the body, such that the surgeons are able to control the direction of the needle position.

In other embodiments, different color sutures are used. For example, the suture 602 has a red color, the suture 604 has a green color, and suture 606 has a yellow color. The different colors of the sutures are able to be easily identified during a surgery.

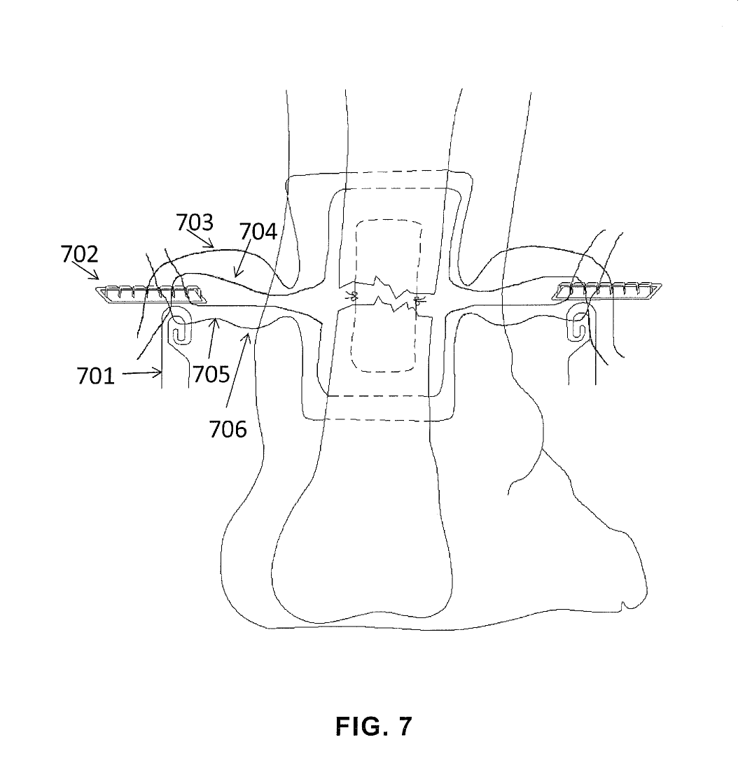

In other embodiments, with the help of the suture collector, a surgeon can easily organize and identify the correct suture to use throughout the surgery. FIG. 7 illustrates an Achilles surgery using the surgical kit with color sutures 703, 704, 705, and 706. Each of the sutures 703, 704, 705 and 706 has a different color. During the surgery, sutures 703, 704, 705 and 706 are inserted into the leg and pulled by a suture retriever 701 one at a time and are placed on the suture collector 702. The surgeon is able to easily identify sutures 703, 704, 705 and 706 with the different colors of the sutures and perform appropriate procedure to each of the sutures.

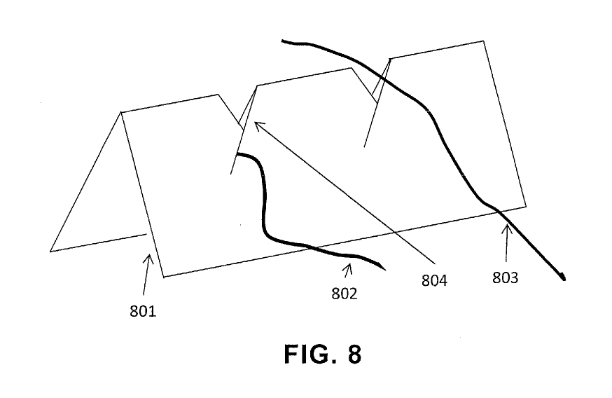

FIG. 8 illustrates a suture collector 801 in accordance with some embodiments. In some embodiments, the suture collectors 801 are made from disposable and foldable material, such as paper. The suture collector 801 is able to be folded in half so the suture collector is able to stands up. The suture 802 is able to be placed in the groove 804 of the Y-shape cut. The groove 804 is able to clamp the suture 802. In contrast, the suture 803 is shown not to be clamped by the suture collector 801.

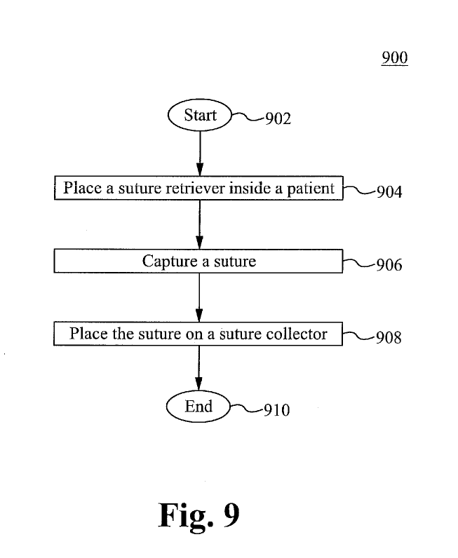

FIG. 9 illustrates a method 900 of using the surgical kit in accordance with some embodiments. The method is able to start from a Step 902. At a Step 904, a suture retriever is placed inside an operational opening of a patient. At a Step 906, the suture retriever is used to capture a suture. At a Step 908, the suture that is captured is placed on a suture collector. The method 900 is able to stop at a Step 910.

In some embodiments, the suture retriever of the surgical kit disclosed herein is able to be made from a disposable material for one time use. This is able to reduce the needs and costs for sensitizing the suture retriever. Since the shape of the suture retriever is altered during the sensitization process in some cases, the use of disposable material is also able to prevent the reuse of the suture retriever.

The surgical kit is able to be utilized in minimally-invasive surgeries. A single surgeon is able to perform a surgery without an assistant by using the surgical kit disclosed herein. In operation, a surgeon is able to place the suture retriever into a wounded area to retrieve a suture. Next, the suture that is retrieved is able to be placed on a predetermined gap (capture hole) on the suture collector to immobilize the sutures.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A surgical kit comprising:
a) a suture retriever comprising a handle element coupling with an extending element, wherein the extending element is coupled with a hook portion; wherein the hook portion comprises a spiral structure and;
b) a suture collector comprising suture collecting elements forming one or more slits configured to receive a suture from the suture retriever and a base coupling both sides of the suture collecting elements.

2. The surgical kit of claim 1, wherein the spiral structure comprises a truncated, a partial, or an abbreviated spiral structure.

3. The surgical kit of claim 1, wherein the spiral structure comprises a coiled structure.

4. The surgical kit of claim 1, wherein the extending element comprises a rectangular structure.

5. The surgical kit of claim 1, wherein the width of the hook portion is the same as the width of the extending element.

6. The surgical kit of claim 5, wherein the width is in the range of 0.3 cm to 0.7 cm.

7. The surgical kit of claim 1, wherein the hook portion comprises a straight shank element extending straight away from the extending element, a first bending element, a first extending element, a second bending element, and a second extending element.

8. The surgical kit of claim 7, wherein the straight shank element is directly connected to the first bending element bended toward the extending element, wherein the first bending element is directly connected to the first extending element that is extended straight toward the extending element.

9. The surgical kit of claim 7, wherein the first extending element is parallel to the straight shank element.

10. The surgical kit of claim 7, wherein the second extending element is parallel to the straight shank element and the first extending element.

11. The surgical kit of claim 7, wherein the straight shank element, the first bending element, the first extending element, the second bending element and the second extending element are formed as an integral piece.

12. The surgical kit of claim 1, wherein the suture retriever or the suture collector is made by hardened steel, tempered steel, stainless steel, high carbon steel or a composition material.

13. The surgical kit of claim 1, wherein the extending element has a thickness same as the thickness of the handle element.

14. The surgical kit of claim 1, wherein the suture collector is made by a disposable material, a foldable material, or a combination thereof.

15. A surgical kit comprising a suture retriever comprising a coiled suture hook on an angled extending arm.

16. The surgical kit of claim 15 further comprising a suture collector having multiple suture receivers to receive sutures from the suture retriever, wherein the sutures comprise different colors.

17. The surgical kit of claim 15 further comprising a needle coupling with only one thread of a suture.

18. A method of using a surgical kit comprising:
a) retrieving a suture using a suture retriever, wherein the suture retriever comprises a handle element coupling with an extending element, wherein the extending element is coupled with a hook portion; wherein the hook portion comprises a spiral structure; and
b) receiving the suture using a suture collector comprising suture collecting elements comprising one or more slits.

19. The method of claim 18, wherein the spiral structure comprises a truncated, a partial, or an abbreviated spiral structure.

20. The method of claim 18, wherein the spiral structure comprises a coiled structure.

21. The method of claim 18 further comprising placing sutures with different colors on the suture collector.
